(19) United States
(12) Patent Application Publication
(10) Pub. No.: US 2010/0137809 A1
(43) Pub. Date: Jun. 3, 2010

(54) INJECTION DEVICE WITH CANNULA SUPPORT WHICH CAN BE CLICKED IN

(76) Inventors: Markus Tschirren, Kirchberg (CH); Ulrich Moser, Heimiswil (CH); Juerg Hirschel, Aarau (CH); Martin Wymann, Liebefeld (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/581,374

(22) Filed: Oct. 19, 2009

(30) Foreign Application Priority Data
Oct. 20, 2008 (DE) DE 102008052356.9

(51) Int. Cl. A61M 5/31

(52) U.S. Cl. 604/187

(57) ABSTRACT

An injection device including a cannula support, an ampoule holder and a holder that holds the cannula support on the ampoule holder, wherein the holder includes an engaging element and a receptacle for the engaging element and is arranged in the region of a side of the cannula support that faces away from the central longitudinal axis of the injection device.

### CROSS-REFERENCE RELATED APPLICATIONS

This application claims priority to German Patent Application DE 10 2008 052 356.9 filed Oct. 20, 2008, the contents of which are incorporated herein by reference.

### BACKGROUND

The invention relates to devices for injecting, administering, delivering, dispensing or infusing a substance, and to methods of making and using such devices. More particularly, it relates to an injection device in which a cannula support is held on a carpoule or ampoule holder of the injection device with the aid of a holder or holding means, wherein at least a part of the holder or holding means is arranged in an outer region and/or in the region of an exterior area of the cannula support.

Threaded engagement systems to fasten a cannula support to an injection device are known from the prior art. In such engagement systems, the cannula support, which comprises an inner thread, is screwed onto a carpoule holder of the injection device which comprises a corresponding or complementary outer thread. Since the thread(s) and the carpoules exhibit standard dimensions, a very thin-walled embodiment of the carpoule holder is necessary in the region of the threaded engagement. This results in problems in the manufacture of the injection-molded carpoule holders, since the molded material has to pass an abrupt tapering of the wall thickness between the thick-walled carpoule holder body and the thin-walled threaded engagement portion of the carpoule holder. For example, in the molding process, plastic flows as far as the constriction during the manufacturing method, then accumulates at said constriction. This problem results in a number of rejects in the production of such carpoule holders.

### SUMMARY

An object of the present invention is to provide an injection device that can be conveniently, efficiently and reliably manufactured using injection molding methods.

In one embodiment, the present invention comprises an injection device comprising a cannula support, an ampoule holder and another holder that holds the cannula support on the ampoule holder, wherein the another holder comprises an engaging element and a receptacle for the engaging element and is arranged in the region of a side of the cannula support that faces away from the central longitudinal axis of the injection device. It should be appreciated that, as used herein, the terms ampoule, carpoule, container, vial, vessel and the like may be thought of and used interchangeably (unless otherwise indicated) to refer to structures and/or devices suitable for containing a substance to be injected, infused or otherwise delivered. The substance may be medicinal or therapeutic.

In one embodiment, the present invention comprises an injection device, comprising a cannula support, a carpoule holder and a holding means which holds the cannula support on the carpoule holder, wherein the holding means comprises an engaging element and a receptacle for the engaging element and is arranged in the region of a side of the cannula support which faces away from the central longitudinal axis (L) of the injection device.

In one embodiment, an injection device in accordance with the present invention comprises a cannula support, a carpoule holder and a holding means for holding the cannula support on the carpoule holder, wherein the holding means comprises at least one engaging element and at least one receptacle which enable a connection between the carpoule holder and the cannula support, wherein the holding means is arranged, as viewed from the cannula support, on the outer side or outside of the cannula support and/or in a region of a side of the cannula support which faces away from the central longitudinal axis of the injection device. In other words, the connection between the receptacle and the engaging element is established in an outer region of the cannula support.

In some embodiments it is conceivable for the cannula support to be slid into the injection device in a distal (front) region of the injection device to be connected to the injection device and/or to a carpoule or ampoule holder associated with the injection device and to then be at least partially surrounded circumferentially by the injection device in the connected position. The cannula support and the carpoule holder are thus connected “from without.” This moves the holding means out of the region between the outer side of the carpoule head or end and the inner side of the cannula support, and the formation of the holding means is no longer bound or limited by the limited available space in this region. It also provides greater freedom for embodying the holding means and/or engaging element and the receptacle, such that the manufacturing method for the holding means and the elements encompassed by it is improved.

Thus, it is possible to avoid the transition between the relatively thick wall thickness of the carpoule holder body and the relatively thin wall thickness of the threaded engagement portion of the carpoule holder, which may be disadvantageous, to enable an embodiment of a carpoule holder which is more conducive to injection molding, wherein the region around the holding means on the carpoule holder can, for example, exhibit an identical or at least similar wall thickness and the carpoule holder can also be embodied to be approximately “cylindrical.” In other words, the holding means on the carpoule holder exhibits approximately the same distance from the central longitudinal axis of the injection device as the wall of the rest of the carpoule holder body.

In one preferred embodiment, the engaging element is formed, e.g. molded, on the carpoule holder and the receptacle is formed, e.g. molded, on the cannula support.

The reverse scenario is, of course, also conceivable, namely that the carpoule holder comprises the receptacle and the cannula support comprises the engaging element. Either way, the holding means is arranged on the outer circumference of the cannula support, and/or the connection between the receptacle and the engaging element is made in the region of the outer circumference of the cannula support.

In another preferred embodiment, the engaging element is an elastically deformable engaging element which can latch on or into the receptacle. In this context, it is thus possible to describe this as a sort of positive-fit connection or friction connection between the engaging element and the receptacle and accordingly between the carpoule holder and the cannula support, as soon as the engaging element latches on or into the receptacle. As soon as the cannula support is “slid into” the carpoule holder, the carpoule holder body can deform the elastically deformable or spring-elastic engaging element to enable the cannula support to be inserted further into the carpoule holder. When a final position of the cannula support relative to the carpoule holder has been reached, the engaging element latches and/or snaps into the receptacle due to its elastic restoring force. The engaging element acting on, engaging with, or at least partially encompassing a receptacle may be thought of and/or referred to as latching, latching in, locking to, or locking with.

In some embodiments, the engaging element moves toward the central longitudinal axis of the injection device as it latches. This can be the case when the receptacle is a cavity on an outer side or outer circumference of the cannula support which is “opened” away from the central longitudinal axis of the injection device and the engaging element comprises a continuation (or extension or protrusion) which is directed toward said central longitudinal axis to engage with and/or act on the receptacle via said continuation. The movement of the engaging element as it latches would, of course, be directed away from the central longitudinal axis if the receptacle is arranged on the inner side of the carpoule holder and the engaging element and/or the continuation of the engaging element is arranged on the outer side of the cannula support. It is also conceivable for the receptacle to be a sort of continuation which is directed away from the central longitudinal axis and arranged on an outer side of the cannula support and which is at least partially encompassed by an engaging element as it latches in, wherein the engaging element can be elastically deformed, about an axis which intersects the central longitudinal axis orthogonally, as it latches in.

An elastically deformable, resilient engaging element can be simply and cost-effectively manufactured from plastic by injection molding and may be integrally molded onto the carpoule holder or the cannula support and thus assigned to or be part of the cannula support or the carpoule holder. Various embodiments are thus conceivable for such an engaging element, for example spring-elastic, resilient, elastically deformable fingers or arms. The engaging element, or engaging elements if a number of engaging elements are used, may extend in the axial direction of the injection device.

In some embodiments, it is also conceivable for the cannula support to be non-releasably held on the carpoule holder by the holding means, i.e. for the hold to be releasable only by destroying one of the support, the holder and/or the holding means. Such an embodiment would help ensure that the cannula support does not detach again from the carpoule holder and/or injection device. Thus, in connection with disposable injection needles which are only intended to be used once, the user is effectively prevented from simply separating the cannula support from the injection device again, to then be used for subsequent injections, since such cannula supports necessarily have to remain on the carpoule holder if a non-releasable connection is provided.

In some embodiments, the receptacle can be a “compact” cavity comprising an edge which is circumferential around the cavity. Using a groove, a heel or a collar and/or an attachment or continuation, or other suitable arrangement as the “receptacle” is also conceivable.

The spring-elastic embodiment of the engaging element has the advantage of signalling to the user, through a typical snap or noise as it latches in, that a connection has been established between the cannula support and the carpoule holder of the injection device and an injection can be securely performed.

In some embodiments, the positive-fit connection between the cannula support and the carpoule holder is a releasable connection, i.e. it is possible for the cannula support to be separated from the carpoule holder again without being destroyed.

In one preferred embodiment, the holding means comprises a separator or separating means for releasing the positive-fit connection. Such a separating means can, for example, be realized in the form of leverage cams on the cannula support and/or carpoule holder which, when the cannula support moves relative to the carpoule holder, slide off on each other and thus generate a leverage movement of the engaging element on or in the receptacle. A combination of leverage cams on one of the components to be connected and serrated leverage elements on the other of the components to be connected is also conceivable. In general, a separating means is intended to generate a movement of the engaging element away from and/or out of the receptacle.

In accordance with another preferred embodiment of the present invention, it is conceivable for the cannula support to be moved translationally and/or rotationally relative to the carpoule holder to release the hold, wherein this movement is caused by the user of the injection device.

In some embodiments, the engaging element may slide or abruptly shoot or move out of the corresponding receptacle. The sort or manner of separation of the engaging element from the receptacle can be influenced by the geometry of the engaging element and the receptacle. Thus, an engaging element which is rounded in the region of the receptacle will slide out of the receptacle, while a cornered and/or sharp-edged engaging element will abruptly shoot out of a corresponding receptacle, as soon as the separating means generates a movement of the engaging element relative to the receptacle.

In some embodiments, the holder or holding means comprises a number of engaging elements and receptacles. Since injection devices and cannula supports are almost exclusively substantially axially symmetrical, the holding means can be embodied such that at least one engaging element always acts on and/or engages with a receptacle in each alignment in which the cannula support is “twisted” about the longitudinal axis of the injection device with respect to the carpoule holder. It is thus possible to help ensure that a secure connection is established when connecting the cannula support to the carpoule holder, irrespective of how the cannula support is “twisted” about the longitudinal axis with respect to the carpoule holder.

In another preferred embodiment, a multitude of engaging elements simultaneously engage with corresponding receptacles, irrespective of the alignment of the cannula support with respect to the carpoule holder.

In some embodiments, the number of engaging elements is greater than the number of receptacles, wherein all or at least one of the engaging elements engage with or act on a number of or at least one receptacle. This may be the case when, for example, a number of engaging elements act on and/or engage with a radially circumferential groove, a radially circumferential heel or a radially circumferential collar as the receptacle.

In some embodiments, a receptacle extending spirally on the circumference of the cannula support or carpoule holder is also conceivable. In this case, it would be possible for the cannula support to be “unscrewed” from the carpoule holder again despite the engaging elements being latched into or on the spirally running receptacle, wherein additional securing elements are conceivable to prevent the cannula support from undesirably detaching from the carpoule holder. For example, a sort of bayonet lock could be provided, such that the engaging element only has to be moved out of the secured position on the receptacle to enable the cannula support to be unscrewed and/or removed from the carpoule holder.

In another preferred embodiment, the engaging element extends parallel to the longitudinal axis of the injection device and is connected to the carpoule holder in the form of a molded-on arm at its end facing away from the distal end of the injection device. The connection between the engaging element and the carpoule holder could also be formed at the end of the engaging element which faces the proximal (rear) end of the injection device. A connection between the engaging element and the carpoule holder at both the distal and the proximal end of the engaging element is also conceivable. In such embodiments, the engaging element may comprise an elastic and/or spring-elastic embodiment.

The engaging element can also be embodied as a groove which extends radially on the inner circumference of the carpoule holder, while the receptacle is a collar which extends radially on the outer circumference of the cannula support, such that the groove moves into the collar and latches in it when the carpoule holder is slid on and/or connected to the cannula support.

### BRIEF DESCRIPTION OF THE DRAWINGS

FIG. 1 depicts an exemplary carpoule holder and injection device;

FIG. 2a depicts a first embodiment of a cannula support which is non-releasably connected to the carpoule holder;

FIG. 2b is a top view onto the distal (front) end of the support of FIG. 2a;

FIG. 3 depicts another embodiment of a cannula support which is non-releasably connected to the carpoule holder;

FIG. 4a depicts one embodiment of a cannula support which is releasably connected to the carpoule holder, in a perspective view;

FIG. 4b is a sectional view of the embodiment of FIG. 4a;

FIG. 5 depicts another embodiment of a cannula support which is releasably connected to the carpoule holder, in a perspective view;

FIG. 6 depicts another embodiment of a cannula support which is releasably connected to the carpoule holder, in a perspective view.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making embodiments of the invention and/or components thereof may be selected from appropriate materials such as metal, metallic alloys, ceramics, plastics, etc. Unless otherwise indicated specifically or by context, positional terms (e.g., up, down, front, rear, distal, proximal, etc.) are descriptive not limiting. Same reference numbers are used to denote same parts or components.

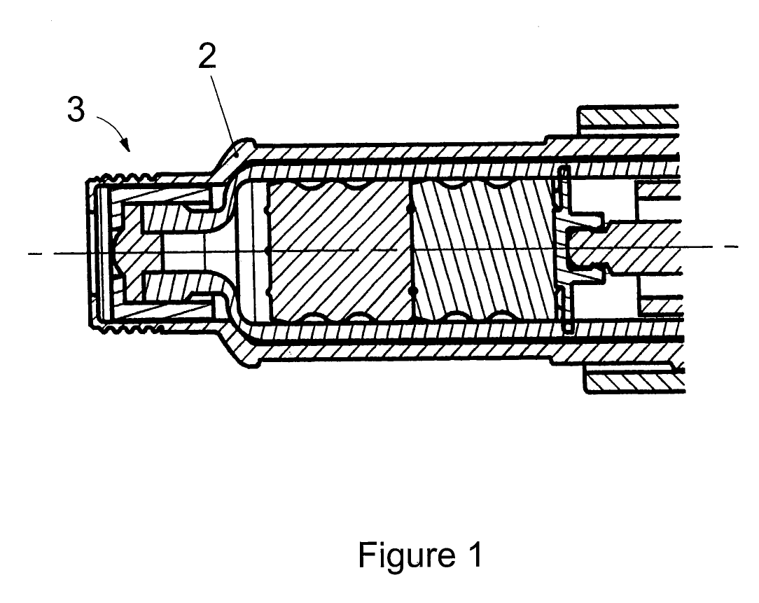

FIG. 1 shows an exemplary injection device in which the carpoule holder 2 comprises a threaded engagement portion 3 at its distal end for connecting the carpoule holder 2 to a cannula support (not shown). It can be seen that in the region of the cross-sectional tapering of the carpoule holder 2, the wall thickness of the carpoule holder 2 abruptly decreases, such that during production, the injection-molded material can only penetrate into the region of the threaded engagement portion 3 with difficulty and there is therefore the danger of a high number of rejects during production.

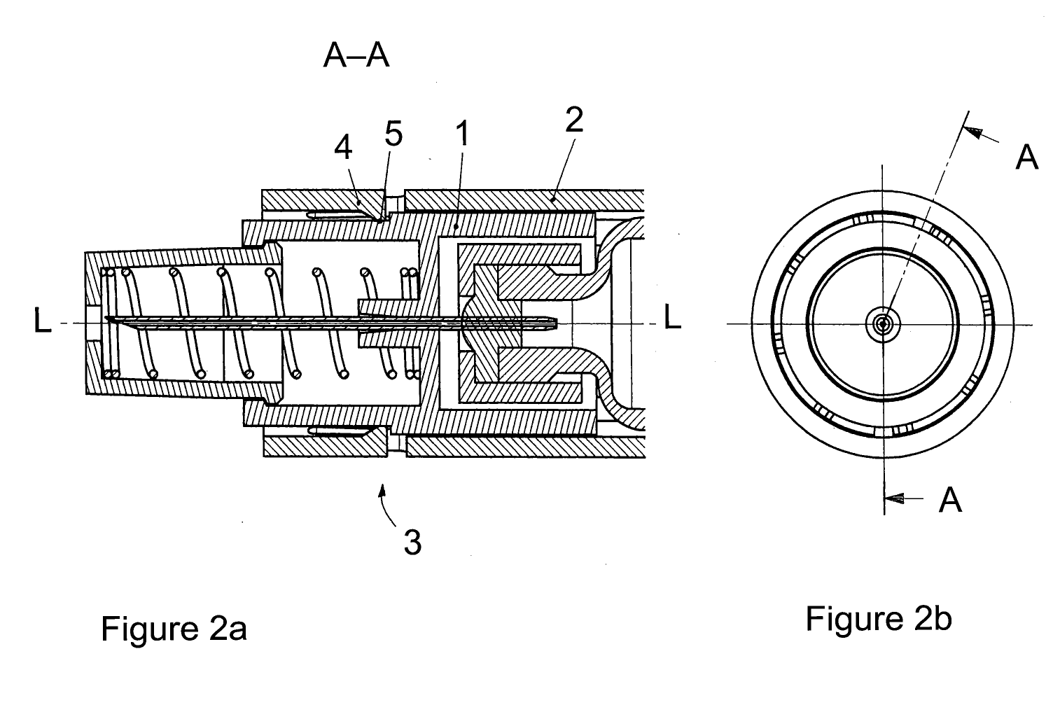

FIG. 2a shows an exemplary carpoule holder 2 in accordance with the present invention which comprises radially circumferential engaging elements 4 at its distal end. These engaging elements 4 are shaped as arms extending parallel to the longitudinal axis L of the injection device and are connected to and/or molded on the carpoule holder 2 at their distal end. They also comprise continuations (or protrusions) which are directed toward the central longitudinal axis L of the injection device and click-in and/or engage behind the circumferential heel 5 of a cannula support 1 when the cannula support 1 is inserted, such that a positive-fit connection between the cannula support 1 and the carpoule holder 2 is established. FIG. 2b shows a top view onto the distal end of the injection device comprising the engaging elements 4 arranged on the circumference of the carpoule holder 2.

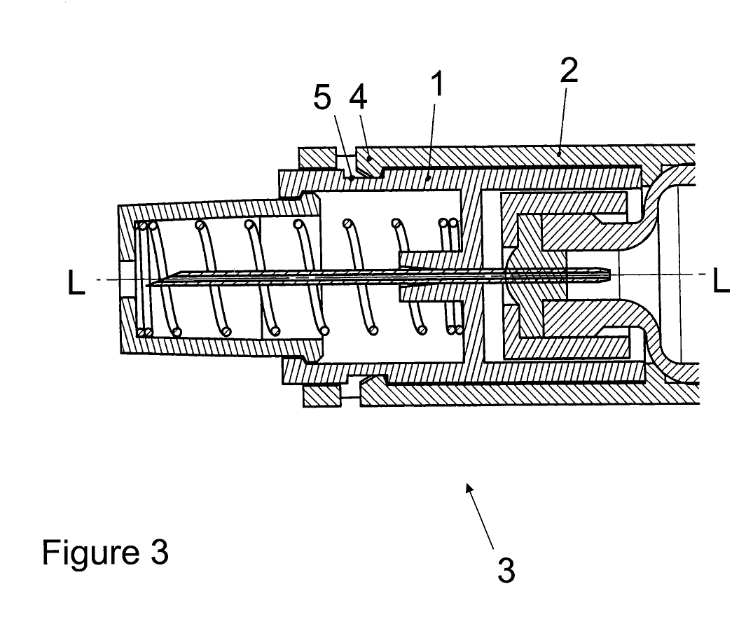

FIG. 3 shows another embodiment of a device in accordance with the present invention, which—like the device shown in FIG. 2a—comprises a non-releasable connection between the carpoule holder 2 and the cannula support 1, wherein the receptacle 5 is formed on the cannula support 1 as a radially circumferential groove, while the engaging elements 4 are not connected to the carpoule holder 2 at their distal end, as in FIG. 2a, but rather via their proximal end. A stopper of the carpoule holder 2 (not identified) can also be seen behind the proximal end of the cannula support 1 and prevents the cannula support 1 from being inserted further into the carpoule holder 2.

In both FIG. 2a and FIG. 3, the regions of the engaging elements 4 which are contacted first by the cannula support 1 when it is inserted into the carpoule holder 2 comprise oblique stop faces such that the engaging elements 4 can recede or be moved away when the cannula support 1 is inserted, to then resile and/or latch-in spring-elastically in the receptacle 5 when the cannula support 1 reaches its final position. In this position, the engaging elements 4 contact the receptacle 5 via substantially more steeply extending flanks, such that the cannula support 1 is effectively prevented from being drawn out of the carpoule holder 2.

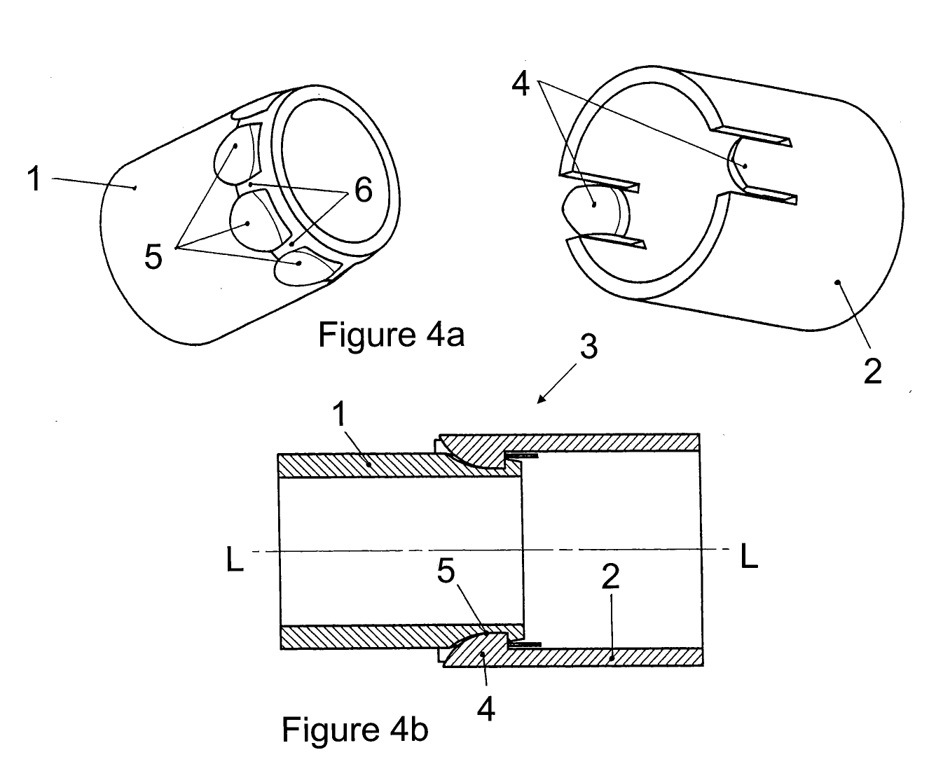

FIG. 4a shows how two engaging elements 4 are arranged on the carpoule holder 2, wherein a number of corresponding receptacles 5 are formed in a uniform distribution on the radial circumference. It can also be seen that the receptacles 5 also comprise areas which are concave in the radially circumferential direction. It is thus possible, simply by twisting the cannula support 1 in the carpoule holder 2, to enable the engaging elements 4 to move out of the receptacles 5 by sliding off on the concave areas of the receptacles 5. The bridges or raised areas 6 between the receptacles 5 on the cannula support 1 can be referred to generally as a separating means, since as soon as the engaging elements 4 respectively come to rest on the bridges 6, the bridges 6 enable the cannula support 1 to be separated from the carpoule holder 2 by drawing or moving the cannula support 1 out of the carpoule holder 2 translationally.

FIG. 4b shows a cannula support 1 comprising receptacles 5 arranged on the outer circumference with which the continuations or protrusions of the engaging elements 4 arranged on the inner circumference of the carpoule holder engage. The engaging elements 4 are connected at their proximal end to the carpoule holder 2 and also comprise convex flanks to cause the engaging elements 4 to yield when the cannula support 1 is inserted into the carpoule holder 2, wherein the convex flanks of the engaging elements 4 transition into a right-angled heel which prevents the positive-fit connection from being released by simply drawing or pulling the cannula support 1 out of the carpoule holder 2.

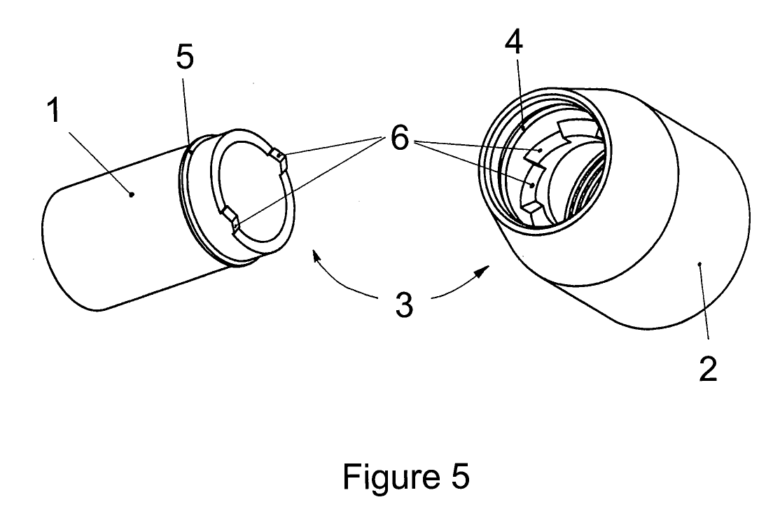

In FIG. 5, the cannula support 1 comprises leverage cams 6 at its proximal end, while the carpoule holder 2 comprises serrated leverage elements 6 in a corresponding region. The leverage cams and the leverage elements may be thought of and/or referred to as a separating means 6. The cannula support 1 also comprises a circumferential collar 5 on its radial circumference, which comes to rest in a corresponding groove on the inner circumference of the carpoule holder 2 when the cannula support 1 is slid into the carpoule holder 2. As soon as this clicked-in, locked and/or latched-in position has been reached, separation is possible by twisting the cannula support 1 with respect to the carpoule holder 2. This is due to the fact that the leverage cams 6 on the cannula support 1 slide off on the serrated flanks of the leverage elements 6 on the carpoule holder 2. A translational movement of the cannula support 1 out of the carpoule holder 2 is thus superimposed on the rotational movement, such that the collar 5 ultimately springs out of the groove 4.

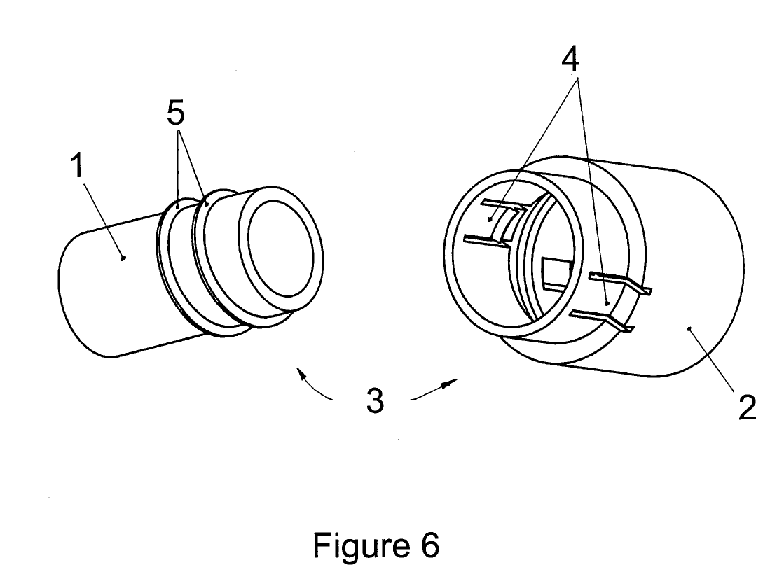

FIG. 6 shows a cannula support 1 comprising a collar 5, which extends spirally on the outer circumference of the cannula support 1, as the receptacle, while the carpoule holder 2 shown comprises two engaging elements which are connected and/or molded to the carpoule holder 2 both at their proximal end and at their distal end. The engaging elements 4 continue to be spring-elastic by being separated from the carpoule holder 2 in the region between the proximal and distal end of the engaging elements 4. Thus, when the cannula support 1 is translationally inserted into the carpoule holder 2, the engaging elements 4 will spring over the collar 5, while the connection between the carpoule holder 2 and the cannula support 1 can be released by simply unscrewing the cannula support 1, since in this case, the engaging elements 4 only slide off on the flanks of the collar 5 which extends spirally.

Embodiments of the present invention, including preferred embodiments, have been presented for the purpose of illustration and description. They are not intended to be exhaustive or to limit the invention to the precise forms and steps disclosed. The embodiments were chosen and described to illustrate the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising a cannula support, an ampoule holder and a holder that holds the cannula support on the ampoule holder, wherein the holder comprises an engaging element and a receptacle for the engaging element and is outside of the cannula support.

2. The injection device in accordance with claim 1, wherein the engaging element is on the ampoule holder and the receptacle is on the cannula support.

3. The injection device in accordance with claim 2, wherein the wall thickness of the ampoule holder is substantially uniform.

4. An injection device comprising a cannula support, an ampoule holder and a holder that holds the cannula support on the ampoule holder, wherein the holder comprises a number of engaging elements and receptacles, wherein at least one engaging element always latches on, in or into a receptacle as the cannula support is turned about a longitudinal axis with respect to the ampoule holder.

5. The injection device in accordance with claim 4, wherein the wall thickness of the ampoule holder is substantially uniform.

6. An injection device, comprising a cannula support, a carpoule holder and a holding means which holds the cannula support on the carpoule holder, wherein the holding means comprises an engaging element and a receptacle for the engaging element and is arranged in the region of a side of the cannula support which faces away from the central longitudinal axis of the injection device.

7. The injection device in accordance with claim 6, wherein the engaging element is on the carpoule holder and the receptacle is on the cannula support.

8. The injection device in accordance with claim 7, wherein the engaging element is an elastically deformable engaging element which can latch on or into the receptacle.

9. The injection device in accordance with claim 8, wherein the engaging element moves toward the central longitudinal axis as it latches.

10. The injection device in accordance with claim 6, wherein the cannula support is non-releasably held on the carpoule holder by the holding means.

11. The injection device in accordance with claim 6, wherein the cannula support is releasably held on the carpoule holder by the holding means.

12. The injection device in accordance with claim 11, wherein the holding means also comprises a separating means by which the hold of the cannula support on the carpoule holder can be released.

13. The injection device in accordance with claim 11, wherein the cannula support is moved translationally and/or rotationally relative to the carpoule holder to release the hold.

14. The injection device in accordance with claim 6, wherein the engaging element is an arm extending parallel to the longitudinal axis of the injection device and is connected to the carpoule holder at its end facing the distal end of the injection device.

15. The injection device in accordance with claim 6, comprising a number of engaging elements and receptacles, wherein at least one of the engaging elements always latches on, in or into a receptacle as the cannula support is twisted about the longitudinal axis with respect to the carpoule holder.

16. The injection device in accordance with claim 6, comprising a number of engaging elements and receptacles, wherein the number of engaging elements is greater than the number of receptacles, and wherein each of the engaging elements latches on or in a single one of the receptacles.

17. The injection device in accordance with claim 6, wherein the receptacle comprises a groove or a collar.

18. The injection device according to claim 17, wherein the groove or collar is radially circumferential on the cannula support.

19. The injection device in accordance with claim 17, wherein the groove or collar extends spirally on the circumference of the cannula support.

20. The injection device in accordance with claim 6, wherein the engaging element is a groove which extends radially on the inner circumference of the carpoule holder and the receptacle is a collar which extends radially on the outer circumference of the cannula support.

21. The injection device in accordance with claim 6, wherein the wall thickness of the carpoule holder in the region of the holding means is substantially the same as the wall thickness of the carpoule holder body generally.
